(19) United States
(12) Patent Application Publication
(10) Pub. No.: US 2026/0226554 A1
(43) Pub. Date: Aug. 6, 2026

(54) METHODS FOR THE PROGNOSIS OF CLEAR CELL RENAL CELL CARCINOMA

(71) Applicants: INSTITUT NATIONAL DE LA SANTÉ ET DE LA RECHERCHE MEDICALE, Paris (FR); UNIVERSITE DE RENNES, Rennes (FR); ÉCOLE DES HAUTES ÉTUDES EN SANTÉ PUBLIQUE, Rennes (FR); ETABLISSEMENT FRANÇAIS DU SANG (EFS), La Plaine Saint Denis (FR); CHU DE RENNES, Rennes (FR)

(72) Inventors: Frédéric CHALMEL, Rennes (FR); Judikael SAOUT, Rennes (FR); Gwendoline LECUYER, Rennes (FR); Nathalie RIOUX, Rennes (FR); Simon LEONARD, Rennes (FR); Aurélie LARDENOIS, Rennes (FR); Bertrand EVRARD, Rennes (FR)

(21) Appl. No.: 19/154,170

(22) PCT Filed: Feb. 9, 2023
(86) PCT No.: PCT/IB2023/000051

§ 371 Date: Aug. 6, 2025

(51) Int. Cl. C12Q 1/6886; C12N 15/10

(52) U.S. Cl. CPC C12Q 1/6886; C12N 15/1065; C12N 15/1096; C12Q 2600/112; C12Q 2600/118; C12Q 2600/158

(57) ABSTRACT

The present invention pertains to a method for the prognosis or clear cell renal cell carcinoma (ccRCC) based on cell clusters identified by deconvolution of a single-cell malignancy atlas. The present inventors have designed and validated a scRNA-seq protocol to characterize the malignant cells (MC) in ccRCC tumors from ccRCC patients. By applying an in silico deconvolution approach 5 to a bulk RNA-seq dataset of ccRCCs, the present inventors have shown that the predicted proportion of specific cells clusters were strongly associated with specific prognoses and notably allow for the accurate stratification of patients. The markers identified by the present inventors particularly allow identifying high-grade patients who would have erroneously been classified as low-grade according to the current classification system.

### TECHNICAL FIELD

The present invention relates to methods for the prognosis of clear cell renal cell carcinoma (ccRCC).

### BACKGROUND ART

Clear cell renal cell carcinoma (ccRCC) is the most frequent and lethal subtype of kidney cancer which represents 2.2% of all cancers and 180,000 deaths annually, worldwide (Sung et al. CA: A Cancer Journal for Clinicians 2021; 71:209-49). It has a poor prognosis, as 45% of diagnosed patients are or become metastatic (Hsieh et al., Nat Rev Dis Primers 2017; 3:17009) and 30% of those treated with surgery eventually relapse, including 10-25% of clinically low-risk patients with progressing disease (called low-risk progressors) (Nerich et al. OncoTargets and Therapy 2014:365; Rini B I. J Clin Oncol 2009; 27:3225-34; Sorbellini et al., J Urol 2005; 173:48-51; Makhov et al., Mol Cancer Ther 2018; 17:1355-64). Recent targeted therapies have provided significant benefits for patients in terms of progression-free and overall survival (Ross K, Jones R J. Clin Sci 2017; 131:2627-42). However, their efficacy is limited with 30% of responsive patients, contrasting responses within tumors, and long-term relapses in most cases (Motzer et al. N Engl J Med 2007; 356:115-24; Molina et al. Eur J Cancer 2014; 50:351-8; Crusz et al. BMC Med 2016; 14:185). These pitfalls are not only due to inter- but also, importantly, to intra-tumor heterogeneity (ITH) which underlies the emergence of novel malignant clones, allowing the tumor to proliferate, escape treatments or relapse after surgery (Lopez J I, Angulo J C. Curr Urol Rep 2018; 19:3; McGranahan N, Swanton C, Cancer Cell 2015; 27:15-26; Raatz et al., PLOS Comput Biol 2021; 17: e1008702; Gerlinger et al, N Engl J Med 2012; 366:883-92). Single-cell RNA sequencing (scRNA-seq) has become the method of choice to decipher expression intra-tumor heterogeneity (eITH) (Suvà ML, Tirosh I. Mol Cell 2019; 75:7-12) by simultaneously profiling the individual transcriptome of thousands of cells from a dissociated tissue. It has contributed to demonstrate that eITH is critical for tumor maintenance and progression (Suvà 2019). In ccRCCs, a pioneering single-cell study provided the first evidence of eITH and revealed differences in targetable signaling pathways between a primary tumor and its metastasis (Kim et al. Genome Biol 2016; 17:80). Other studies have used scRNA-seq to explore renal carcinomas, providing new insights into tumor subtypes, origin and differentiation of RCC cells (see e.g. Su et al., Front Oncol 2021; 11:719564) or characterization of the immune landscape (Bi et al., Cancer Cell 2021; 39:649-61.e5). Most of the datasets used by these studies comprise a large proportion of immune cells (ICs) and only a small fraction of malignant cells (MCs).

Hence, there is still a need for single-cell atlases focusing on MC heterogeneity which might provide new insights into the ability of ccRCC to proliferate, escape treatments, metastasize, or relapse. Such atlases could advantageously be used for developing novel and more accurate methods for determining the prognosis of ccRCC, thereby allowing for better patient management.

### SUMMARY

The invention is defined by the claims.

The present invention pertains to a method for the prognosis of clear cell renal cell carcinoma (ccRCC) in a patient, said method comprising the following steps:
a. Obtaining a bulk transcriptomic analysis of a tumor sample obtained from said patient;
b. Submitting said bulk transcriptomic analysis to a deconvolution step so as to determine the proportion, in the tumor sample, of cells belonging to each of the 18 cells clusters consisting of Malignant cells_c3, Malignant cells_c10, Malignant cells_c23, Malignant cells_c9, Malignant cells_c8, Malignant cells_others, B lymphocytes, Collecting duct epithelial cells, Endothelial cells, Erythrocytes, Fibroblasts, Macrophages, Mast cells, NK cells, Pericytes, Plasma cells, T lymphocytes and Tubular epithelial cells;
c. Providing a prognosis based on said cell proportion.

According to a specific embodiment, the transcriptomic analysis can be obtained by submitting the tumor sample to bulk RNA sequencing (RNA-seq) or to bulk RNA barcoding and sequencing (BRB-seq).

According to the present invention, the cell cluster referred to as Malignant cells_c8 is associated with a poor prognosis, and the cell cluster referred to as Malignant cells_c9 is associated with a good prognosis.

### BRIEF DESCRIPTION OF DRAWINGS

FIG. 1: Kaplan-Meier survival curves of clinically low-risk patients (T1-T2) according to their high (dashed curves) or low (curves) prevalence of the “Malignant cells_c8” (A) and of the “Malignant cells_c9” (B), the relative prevalence of the “Malignant cells_c8 (D) and of the “Malignant cells_c9” (E), the differential prevalence between the “Malignant cells_c9” and the “Malignant cells_c8” (C), the relative differential prevalence between the “Malignant cells_c9” and the “Malignant cells_c8” (F). The analysis was performed on lower risk (T1-T2) patients. The corresponding log rank p values are indicated on top of each survival curve.

### DETAILED DESCRIPTION OF THE INVENTION

The present inventors have designed and validated a scRNA-seq protocol to characterize the cells populations in ccRCC tumors from patients ranging from pT1a to pT3b, which was complemented with a scRNA-seq dataset composed of three pairs of ccRCC and adjacent normal kidney samples (Young, et al. Science 2018; 361:594-9). The subsequent analysis revealed contrasted degrees of heterogeneity of malignant cells (MC) within tumors. One particular tumor displayed extensive clonal diversity of MC, recapitulating aggressive features linked to epithelial-mesenchymal transition, cell migration, angiogenesis and cell adhesion. By applying an in silico deconvolution approach to a bulk RNA-seq dataset of ccRCCs, the present inventors have shown that the predicted proportion of specific cells clusters were strongly associated with specific prognoses and notably allow for the accurate stratification of patients. The cell clusters identified by the present inventors particularly allow identifying clinically low-risk patients who will relapse (also called low-risk progressors), from a biopsy of their primary tumor.

The inventors have therefore identified specific cell clusters that were shown to be strongly associated with tumor progression in ccRCC based on a single-cell malignancy atlas. The present inventors have further shown that it is possible to specifically provide a prognosis for ccRCC patients by applying a deconvolution to transcriptomic data determined in the patient's tumor, so as to determine the proportion of each cell subpopulation present within the tumor. The proportion of cells belonging to each cluster is representative of the patient's prognosis.

Therefore, according to a first embodiment, the present invention pertains to a method for the prognosis of clear cell renal cell carcinoma (ccRCC) in a patient, said method comprising the following steps:
a. Obtaining a bulk transcriptomic analysis of a tumor sample obtained from said patient;
b. Submitting said bulk transcriptomic analysis to a deconvolution step so as to determine the proportion, in the tumor sample, of cells belonging to each of the 18 cells clusters consisting of Malignant cells_c3, Malignant cells_c10, Malignant cells_c23, Malignant cells_c9, Malignant cells_c8, Malignant cells_others, B lymphocytes, Collecting duct epithelial cells, Endothelial cells, Erythrocytes, Fibroblasts, Macrophages, Mast cells, NK cells, Pericytes, Plasma cells, T lymphocytes and Tubular epithelial cells;
c. Providing a prognosis based on said cell proportion.

“Clear cell renal cell carcinoma” or “ccRCC” is the most aggressive subtype of kidney cancer. In adults, ccRCC is the most common type of kidney cancer, representing around 80% of all renal cell carcinoma cases. ccRCC is staged according to the TNM (for “Tumor-Node-Metastasis”) staging system which is considered as the gold standard method for staging cancer by the Union for International Cancer Control. The TNM staging system uses the size of the tumor, the presence or absence of tumor in regional lymph nodes, and the presence or absence of distant metastases, to assign a stage and an outcome to the tumor. It was developed from the observation that patients with small tumors have better prognosis than those with tumors of greater size at the primary site. In general, patients with tumors confined to the primary site have better prognosis than those with regional lymph node involvement, which in turn is better than for those with distant spread of disease from one body part to another. Accordingly, cancers are usually staged into four levels. Stage I cancer is a localized cancer with no cancer in the lymph nodes. Stage II cancer has spread near to where the cancer started. Stage III cancer has spread to lymph nodes. Stage IV cancer has spread to a distant part of the body. TNM classification for renal cell carcinoma further divides each of stages I to III in sub-stages qualified as stages pT1a, pT1b, pT2a, pT2b, and pT3a. pT1a (for “primary tumor subtype 1a”) is defined as a stage I tumor that is less than or equal to 4 cm and has not metastasized, pT1b refers to non-metastasized tumors between 4 and 7 cm, pT2a to tumors between 7 cm and 10 cm, pT2b to tumor larger than 10 cm but limited to the kidney and pT3a represents a locally advanced disease with invasion of the fat or vasculature of the kidney. The assigned stage is used as a basis for selection of appropriate therapy and for prognostic purposes. ccRCC treatment usually involves radical or partial nephrectomy as well as immunotherapy, but even when renal tumor resection is carried out during the early stages of tumor development, 20-30% of patients still develop local or distal metastases (Wang et al., Frontiers in Oncology 11 (2021): 543854). These patients, who were stratified as having a low-grade tumor (stage I/II) are ultimately classified as having high-grade ccRCC and, as such, require aggressive treatments and extensive follow-up. The method according to the present invention allows identifying clinically low-risk patients who have a risk of relapse (also called low-risk progressors). Therefore, it is particularly advantageous when the tumor is classified as a low-grade tumor, i.e. as a stage I or II tumor according to the TNM staging system.

Accordingly, according to a specific embodiment, the method according to the present invention is applied to a tumor classified as being a stage I or II tumor according to the TNM staging system.

The “tumor sample” used in the context of the present invention is typically obtained by means of a primary tumor biopsy or during surgery for patients undergoing either radical or partial nephrectomy.

The method according to the present invention comprises obtaining a bulk transcriptomic analysis of a tumor sample. A “bulk transcriptomic analysis” has its general meaning in the art. It refers to the study of the complete set of RNA transcripts that are produced in the tumor sample. This analysis therefore aims at quantifying the expression level of each RNA transcript, including coding and non-coding, in the tumor sample so as to reflect the genes that are being actively expressed in the tumor at a given time. The skilled person knows multiple techniques that allow for obtaining transcriptomics dataset. Transcriptomics is a quantitative science that encompasses the assignment of a list of strings (“reads”) to the object (“transcripts” in the genome). To calculate the expression strength, the density of reads corresponding to each object is counted (Cellerino, Alessandro, and Michele Sanguanini. Transcriptome Analysis: Introduction and Examples from the Neurosciences. Vol. 17. Springer, 2018). Currently, the three main transcriptomics techniques used include DNA microarrays, RNA sequencing (RNA-seq) and bulk RNA barcoding and sequencing (BRB-seq). These techniques require RNA isolation through RNA extraction techniques, followed by its separation from other cellular components and enrichment of mRNA.

According to the present invention, the transcriptomic analysis is preferably obtained by submitting the tumor sample to RNA-seq or to BRB-seq.

“RNA sequencing” or “RNA-seq” is a next-generation sequencing technology that allows for both qualitative and quantitative analysis of RNA transcripts. All RNA-seq methods are based on the use of a reverse transcriptase to convert the RNA to cDNA. It involves RNA extraction, RNA fragmentation, reverse transcription into cDNA, library amplification, and sequencing on a platform to get strings of continuous sequence data in “reads”. RNA-seq is routinely used for transcriptomic analysis, and multiple publications on this topic are available (see e.g. Conesa et al., Genome biology 17.1 (2016): 1-19; or Haque et al (Genome medicine 9.1 (2017): 1-12)). RNA-seq platforms can be easily obtained from various specialized manufacturers.

“Bulk RNA barcoding and sequencing” or “BRB-seq” is one of the most recent techniques developed for optimized transcriptomic analysis. The specificity of BRB-seq technology is that it uses highly optimized barcoded oligo (dT) primers that uniquely tag each individual RNA sample during the first-strand synthesis step of cDNA library preparation. This first-strand synthesis step is the first step of BRB-seq protocol. It consists in the synthesis of a first cDNA strand via reverse transcription and generating barcoded samples with unique molecular identifiers (UMIs). During this step, individual barcoded samples are pooled, free primers are digested, and double-stranded full-length cDNA is generated and subsequently purified with solid-phase reversible immobilization (SPRI) beads. Purified cDNA is then subject to a process called “tagmentation”, in which double-stranded, full-length cDNA is fragmented and tagged using Tn5 transposase, which cleaves the cDNA and ligates adaptors for library amplification (Picelli et al. Genome Research 24 (12): 2033-40). The resulting cDNA library is then indexed and amplified. After purification, the final cDNA library is sequenced (for review, see Alpern et al. Genome biology 20.1 (2019): 1-15). BRB-seq is particularly advantageous as it is quick, cost efficient and allows preserving brand specificity.

The method according to the invention comprises a step wherein bulk transcriptomics dataset, i.e. the results of the transcriptomic analysis of the tumor, are submitted to a deconvolution step using, as a reference, the scRNA-seq dataset composed of a normalized expression matrix file and a cell annotation file. The normalized expression matrix file (named GSE224630_normalized_integrated.data.tsv.gz) and the associated cell annotation file (named GSE224630_normalized_integrated.metadata.tsv.gz) have been made available to the public via the international repository GEO (ncbi.nlm.nih.gov/geo website) under the accession number GSE224630. Thus, in the context of the present invention, the deconvolution step is performed by using the data (the normalized expression matrix file and the associated cell annotation file) deposited as accession number GSE224630 in the National Center for Biotechnology Information Gene Expression Omnibus (NCBI/GEO) as a reference for deconvolution.

“Deconvolution”, or more specifically “cellular deconvolution” refers to a computational technique aiming at estimating the proportions of different cell types in samples collected from a tissue. Deconvolution utilizes scRNA-seq data and cell annotation data to estimate cell type proportions in a bulk transcriptomics sample (RNA-seq or BRB-seq). It therefore allows determining the proportion of each cell type described in the scRNA-seq dataset in a bulk transcriptomics sample (RNA-seq or BRB-seq). In the context of the present invention, the scRNA-seq data and cell annotation used as a reference for performing the step of deconvolution are the files available under reference GSE224630 in the NCBI/GEO international database. 25 Deconvolution can be applied by using various deconvolution tools that are easily available to the skilled person. In the context of the present invention, deconvolution can e.g. performed by using the MuSiC R package (version 0.2.0). Briefly, the scRNA-seq data (normalized expression matrix file and the associated cell annotation file) and the bulk transcriptomics dataset are loaded into two distincts ExpressionSet objects in R, named sc.data and bulk.data, respectively. The cellular deconvolution is then performed using the music_prop function implemented into the MuSiC package. The relative predicted proportion of each malignant cell clusters (including “Malignant cells_c8”, “Malignant cells_c9”, “Malignant cells_c10”, “Malignant cells_c23”, “Malignant cells_c3” and “Malignant cells_others”) is next calculated by dividing the overall predicted proportion of each malignant cell cluster by the overall predicted proportion of malignant cells (corresponding to the sum of all predicted proportions of malignant cell clusters) within a given bulk transcriptomics sample.

The deconvolution step allows estimating cell type proportions in the tumor sample from bulk RNA-sequencing data using, as reference, specific cell clusters (i.e. cell groups) obtained from single-cell RNA-seq data. The cell clusters according to the present invention are Malignant cells_c3, Malignant cells_c10, Malignant cells_c23, Malignant cells_c9, Malignant cells_c8, Malignant cells_others, B lymphocytes, Collecting duct epithelial cells, Endothelial cells, Erythrocytes, Fibroblasts, Macrophages, Mast cells, NK cells, Pericytes, Plasma cells, T lymphocytes and Tubular epithelial cells. In the context of the present invention, the deconvolution will therefore predict the cell type proportions among those specific cell clusters that were defined based on the scRNA-seq.

Each cell cluster according to the present invention represents a homogeneous group of cells assembled based on polyA+transcript expression. Clustering is e.g. independent of assumptions about lineage. Each cluster includes marker genes, but, because the number of genes sampled is large, the identity of each cluster as a “cell type” may or may not depend on that marker. As mentioned above, the method according to the present determines the proportion of cells belonging to each of the 18 cell clusters defined.

In the context of the present invention, “Malignant cells_c3” is an expression used for referring to a group of malignant cells. Cells are clustered in this group mostly based on the expression of the 10 genes COPS2, COQ4, TAF9, PFDN6, SLC17A4, EBLN3P, POLE4, LETMD1, ACE2 and SLC5A10. The expression of these 10 genes is the most prominent feature for assigning a cell to this specific cluster.

In the context of the present invention, “Malignant cells_c10” is an expression used for referring to a group of malignant cells which are mostly defined based on the expression of the 10 genes ZNF667-AS1, MXI1, ALPK2, CDIPT, NME5, ANK3, RNLS, TSEN34, LYG1 and TNFRSF21. The expression of these 10 genes is the most prominent feature for assigning a cell to this specific cluster. This cluster of cells was found to be enriched in genes associated with cellular response to cytokine stimuli (34, 1.5e-3) and response to type I interferon (9, 1.2e-2), indicating innate immune activity.

In the context of the present invention, “Malignant cells_c23” is an expression used for referring to a group of malignant cells which are characterized by organic acid (39, 3.4e-3) metabolic process, secretion (46, 1.8e-2) and programmed cell death (59, 1.3e-2) biological processes. Cells of this group are mostly defined based on the expression of the 10 genes OTULINL, SULT1C2, C16orf74, TSPAN9, MRPL42, PABPN1, SERPINA6, HNRNPAB, BET1 and AC103702.2. The expression of these 10 genes is the most prominent feature for assigning a cell to this specific cluster.

In the context of the present invention, “Malignant cells_c9” is an expression used for referring to a malignant group of cells which are mostly defined based on the expression of the 10 genes HLA-G, MUC1, MRPL12, ALPL, RIMKLA, GRPEL1, CXCL6, ADPRHL2, NAPRT and FH. The expression of these 10 genes is the most prominent feature for assigning a cell to this specific cluster. This cell cluster was significantly associated with cytokine-mediated signaling pathways (22, 6.5e-3), antigen processing and presentation (11, 8.2e-3), and more precisely T cell mediated cytotoxicity (5, 1.7e-2) suggesting the organization of an adaptive immune response in c9. These cells are also characterized by genes involved in programmed cell death (42, 3.9e-3), and are further linked to cell adhesion (34, 3.3e-3), secretion (30, 3.2e-2), and cellular responses to hypoxia (10, 1.0e-2), the initiation stage of angiogenesis [29].

In the context of the present invention, “Malignant cells_c8” is an expression used for referring to a group of malignant cells which are mostly defined based on the expression of the 10 genes AC092336.1, AC079298.3, EFNA5, ING2, SPINK1, PPP1R13L, EFCAB2, CCDC74A, CYHR1 and SSBP2. The expression of these 10 genes is the most prominent feature for assigning a cell to this specific cluster.

In the context of the present invention, “Malignant cells_others” is an expression used for referring to a group of cells which are mostly defined based on the expression of the 10 genes TOP2A, NUSAP1, UBE2C, BIRC5, DTYMK, UBE2T, MAD2L1, CCDC34, PTTG1 and PCNA. The expression of these 10 genes is the most prominent feature for assigning a cell to this specific cluster.

In the context of the present invention the cluster “B Lymphocytes” refers to a group of cells sharing the expression markers usually representative of B lymphocytes. The two most prominent markers for assigning a cell to this cluster are the expression of genes IGHA1 and IGHA2.

In the context of the present invention the cluster “Collecting duct epithelial cells” refers to a group of cells sharing the expression markers usually expressed by collecting duct epithelial cells, i.e. cells forming the epithelial layer lining the collecting duct. The 10 most prominent markers for assigning a cell to this cluster in the context of the present invention are the expression of genes C8orf4, WBP5, KNG1, HSPA2, DUSP9, UMOD, S100A2, TMEM52B, KRT7 and DMKN.

In the context of the present invention the cluster “Endothelial cells” refers to a group of cells sharing the expression markers usually expressed by the cells lining the endothelium, i.e. the interior surface of blood vessels and lymphatic vessels. The 10 most prominent markers for assigning a cell to this cluster in the context of the present invention are the expression of genes RAMP2, RNASE1, SLC9A3R2, FLT1, PLVAP, NOTCH4, HYAL2, EGFL7, HSPG2 and ADGRL4.

In the context of the present invention the cluster “Erythrocytes” refers to a group of cells sharing the expression markers usually representative of red blood cells. The two most prominent markers for assigning a cell to this cluster in the context of the present invention are the expression of genes HBD and SLC25A39.

In the context of the present invention the cluster “Fibroblasts” refers to a group of cells sharing the expression markers usually expressed by this type extracellular matrix- and collagen-producing cells forming a large part of the connective tissue in animals. The 10 most prominent markers for assigning a cell to this cluster in the context of the present invention are the expression of genes DCN, FBLN1, CCDC80, PRELP, ISLR, LUM, FBLN2, SFRP4, MFAP4, and CTSK.

In the context of the present invention the cluster “Macrophages” refers to a group of cells sharing the expression markers usually expressed by this type of white blood cell of the innate immune system that allow destroying pathogens by phagocytosis. The 10 most prominent markers for assigning a cell to this cluster in the context of the present invention are the expression of genes LYZ, AIF1, LST1, MNDA, MS4A7, MS4A6A, SPI1, CSTA, CLEC7A and S100A9.

In the context of the present invention the cluster “Mast cells” refers to a group of cells sharing the expression markers usually expressed by mastocytes which are a type of granulocytes present in the connective tissue and comprising granules rich in histamine and heparin. The 10 most prominent markers for assigning a cell to this cluster in the context of the present invention are the expression of genes TPSAB1, TPSB2, CPA3, MS4A2, HPGDS, SLC18A2, VWA5A, KIT, GATA2 and IL1RL1.

In the context of the present invention the cluster “NK cells” refers to a group of cells sharing the expression markers usually expressed by natural killer cells which are a type of cytotoxic lymphocyte. The 10 most prominent markers for assigning a cell to this cluster in the context of the present invention are the expression of genes KLRD1, GNLY, GZMB, PRF1, FGFBP2, KLRF1, GZMH, PLAC8, CLIC3 and GZMM.

In the context of the present invention the cluster “Pericytes” refers to a group of cells sharing the expression markers usually expressed by this type of multi-functional mural cells of the microcirculation that wrap around the endothelial cells lining the capillaries. The 10 most prominent markers for assigning a cell to this cluster in the context of the present invention are the expression of genes RGS5, PLN, TPPP3, GJA4, MYH11, NOTCH3, ISYNA1, COX412, C11orf96 and CLMN.

In the context of the present invention the cluster “Plasma cells” refers to a group of cells sharing the expression markers usually expressed by this type of antigen-producing white blood cells deriving from lymphoid organs. The 10 most prominent markers for assigning a cell to this cluster in the context of the present invention are the expression of genes IGHG1, IGHGP, IGHG2, JSRP1, IGLV2-23, ST6GAL1, ITGB7, MEI1, VSIR and MAN1A1.

In the context of the present invention the cluster “T-Lymphocytes” refers to a group of cells sharing the expression markers usually expressed by this group of white blood cells characterized by the expression of a T-cell receptor on their surface. The most prominent markers for assigning a cell to this cluster in the context of the present invention are the expression of genes TRAC, GZMK, LTB, DUSP4, KIAA1551, SPOCK2, CD8B, LINC00152 and GPR171.

In the context of the present invention the cluster “Tubular epithelial cells” refers to a group of cells sharing the expression markers usually expressed by cells of the tubulointerstitium. The 10 most prominent markers for assigning a cell to this cluster in the context of the present invention are the expression of genes ALDOB, MIOX, HPD, PCK1, ASS1, FABP1, MT1H, HRSP12, FBP1 and ALDH6A1.

According to the present invention, the genes disclosed in Table 1 below characterize the cell clusters identified by scRNA-seq. Each of the genes disclosed in this Table is known to the skilled person who can easily retrieve the corresponding sequence in all major databases.

TABLE 1
gene markers representative of each gene cluster according to the invention

| | | Mean | logFC:Fold- | | |
|---|---|---|---|---|---|
| | | expression | change ratio | Diff.pct: | |
| | | of the | between | Percentage | |
| | | gene in | the cells | of cells of | |
| | | the cells | of the cluster | said | |
| | | belonging | and the other | cluster | |
| | | to said | cells of | expressing | |
| Genes | Cell cluster | cluster | the dataset | said gene | adjusted.p |
| COPS2 | Malignant cells_c3 | 0.561 | 0.375 | 40.3 | 3.0E−40 |
| COQ4 | Malignant cells_c3 | 0.443 | 0.289 | 36.6 | 1.4E−36 |
| TAF9 | Malignant cells_c3 | 0.467 | 0.318 | 36.5 | 1.5E−37 |
| PFDN6 | Malignant cells_c3 | 0.498 | 0.318 | 35.4 | 8.2E−32 |
| SLC17A4 | Malignant cells_c3 | 0.299 | 0.289 | 34.2 | 2.6E−284 |
| EBLN3P | Malignant cells_c3 | 0.325 | 0.266 | 33.1 | 9.1E−58 |
| POLE4 | Malignant cells_c3 | 0.512 | 0.252 | 32.6 | 1.8E−19 |
| LETMD1 | Malignant cells_c3 | 0.473 | 0.331 | 32.3 | 2.4E−32 |
| ACE2 | Malignant cells_c3 | 0.288 | 0.256 | 31.5 | 9.6E−122 |
| SLC5A10 | Malignant cells_c3 | 0.292 | 0.253 | 30.7 | 3.6E−107 |
| ZNF667- | Malignant cells_c10 | 0.497 | 0.400 | 43.7 | 0.0E+00 |
| AS1 | | | | | |
| MXI1 | Malignant cells_c10 | 0.474 | 0.387 | 40.8 | 0.0E+00 |
| ALPK2 | Malignant cells_c10 | 0.389 | 0.371 | 40.3 | 0.0E+00 |
| CDIPT | Malignant cells_c10 | 0.571 | 0.387 | 40.1 | 7.5E−191 |
| NME5 | Malignant cells_c10 | 0.369 | 0.330 | 40.0 | 0.0E+00 |
| ANK3 | Malignant cells_c10 | 0.416 | 0.359 | 39.6 | 0.0E+00 |
| RNLS | Malignant cells_c10 | 0.466 | 0.438 | 38.5 | 0.0E+00 |
| TSEN34 | Malignant cells_c10 | 0.500 | 0.336 | 38.3 | 1.6E−184 |
| LYG1 | Malignant cells_c10 | 0.364 | 0.345 | 37.3 | 0.0E+00 |
| TNFRSF21 | Malignant cells_c10 | 0.376 | 0.323 | 37.0 | 0.0E+00 |
| OTULINL | Malignant cells_c23 | 0.706 | 0.689 | 65.9 | 0.0E+00 |
| SULT1C2 | Malignant cells_c23 | 0.487 | 0.446 | 60.3 | 5.8E−158 |
| C16orf74 | Malignant cells_c23 | 0.545 | 0.515 | 54.1 | 4.0E−150 |
| TSPAN9 | Malignant cells_c23 | 0.545 | 0.472 | 54.0 | 3.7E−70 |
| MRPL42 | Malignant cells_c23 | 0.605 | 0.392 | 50.4 | 8.7E−25 |
| PABPN1 | Malignant cells_c23 | 0.610 | 0.429 | 50.0 | 1.1E−28 |
| SERPINA6 | Malignant cells_c23 | 0.514 | 0.484 | 48.2 | 2.6E−152 |
| HNRNPAB | Malignant cells_c23 | 0.702 | 0.471 | 47.3 | 4.7E−23 |
| BET1 | Malignant cells_c23 | 0.485 | 0.364 | 47.2 | 5.1E−33 |
| AC103702.2 | Malignant cells_c23 | 0.378 | 0.368 | 46.6 | 1.5E−239 |
| HLA-G | Malignant cells_c9 | 0.608 | 0.572 | 58.8 | 0.0E+00 |
| MUC1 | Malignant cells_c9 | 0.557 | 0.536 | 51.5 | 0.0E+00 |
| MRPL12 | Malignant cells_c9 | 0.670 | 0.496 | 51.1 | 0.0E+00 |
| ALPL | Malignant cells_c9 | 0.457 | 0.415 | 47.0 | 0.0E+00 |
| RIMKLA | Malignant cells_c9 | 0.382 | 0.369 | 46.9 | 0.0E+00 |
| GRPEL1 | Malignant cells_c9 | 0.505 | 0.356 | 45.4 | 6.8E−290 |
| CXCL6 | Malignant cells_c9 | 0.526 | 0.520 | 45.4 | 0.0E+00 |
| ADPRHL2 | Malignant cells_c9 | 0.425 | 0.346 | 45.0 | 0.0E+00 |
| NAPRT | Malignant cells_c9 | 0.550 | 0.355 | 44.6 | 3.2E−224 |
| FH | Malignant cells_c9 | 0.448 | 0.339 | 44.2 | 0.0E+00 |
| AC092336.1 | Malignant cells_c8 | 0.500 | 0.490 | 53.4 | 0.0E+00 |
| AC079298.3 | Malignant cells_c8 | 0.409 | 0.404 | 49.0 | 0.0E+00 |
| EFNA5 | Malignant cells_c8 | 0.449 | 0.434 | 48.8 | 0.0E+00 |
| ING2 | Malignant cells_c8 | 0.500 | 0.427 | 48.2 | 0.0E+00 |
| SPINK1 | Malignant cells_c8 | 0.418 | 0.408 | 46.4 | 0.0E+00 |
| PPP1R13L | Malignant cells_c8 | 0.413 | 0.378 | 45.7 | 0.0E+00 |
| EFCAB2 | Malignant cells_c8 | 0.395 | 0.346 | 45.1 | 0.0E+00 |
| CCDC74A | Malignant cells_c8 | 0.368 | 0.346 | 44.9 | 0.0E+00 |
| CYHR1 | Malignant cells_c8 | 0.466 | 0.347 | 43.6 | 0.0E+00 |
| SSBP2 | Malignant cells_c8 | 0.438 | 0.345 | 43.3 | 0.0E+00 |
| TOP2A | Malignant cells_others | 0.441 | 0.432 | 26.4 | 4.1E−139 |
| NUSAP1 | Malignant cells_others | 0.440 | 0.410 | 25.5 | 1.9E−35 |
| UBE2C | Malignant cells_others | 0.443 | 0.436 | 25.1 | 8.5E−150 |
| BIRC5 | Malignant cells_others | 0.330 | 0.323 | 25.1 | 3.6E−141 |
| DTYMK | Malignant cells_others | 0.422 | 0.338 | 24.2 | 1.2E−13 |
| UBE2T | Malignant cells_others | 0.339 | 0.319 | 23.7 | 6.7E−46 |
| MAD2L1 | Malignant cells_others | 0.356 | 0.331 | 23.4 | 2.8E−39 |
| CCDC34 | Malignant cells_others | 0.334 | 0.288 | 22.2 | 2.2E−18 |
| PTTG1 | Malignant cells_others | 0.591 | 0.522 | 20.0 | 3.3E−14 |
| PCNA | Malignant cells_others | 0.406 | 0.280 | 15.7 | 1.4E−05 |
| IGHA1 | B lymphocytes | 5.289 | 5.213 | 73.9 | 0.0E+00 |
| IGHA2 | B lymphocytes | 2.998 | 2.993 | 63.0 | 0.0E+00 |
| C8orf4 | Collecting duct epithelial | 1.007 | 0.946 | 35.7 | 0.0E+00 |
| | cells | | | | |
| WBP5 | Collecting duct epithelial | 0.921 | 0.816 | 34.2 | 0.0E+00 |
| | cells | | | | |
| KNG1 | Collecting duct epithelial | 0.958 | 0.947 | 33.7 | 0.0E+00 |
| | cells | | | | |
| HSPA2 | Collecting duct epithelial | 0.886 | 0.834 | 31.7 | 0.0E+00 |
| | cells | | | | |
| DUSP9 | Collecting duct epithelial | 0.767 | 0.765 | 29.5 | 0.0E+00 |
| | cells | | | | |
| UMOD | Collecting duct epithelial | 0.868 | 0.825 | 27.5 | 0.0E+00 |
| | cells | | | | |
| S100A2 | Collecting duct epithelial | 0.715 | 0.698 | 27.2 | 0.0E+00 |
| | cells | | | | |
| TMEM52B | Collecting duct epithelial | 0.853 | 0.843 | 25.5 | 0.0E+00 |
| | cells | | | | |
| KRT7 | Collecting duct epithelial | 0.600 | 0.576 | 23.3 | 0.0E+00 |
| | cells | | | | |
| DMKN | Collecting duct epithelial | 0.434 | 0.408 | 22.2 | 9.6E−240 |
| | cells | | | | |
| RAMP2 | Endothelial cells | 2.030 | 1.999 | 76.0 | 0.0E+00 |
| RNASE1 | Endothelial cells | 1.987 | 1.935 | 74.7 | 0.0E+00 |
| SLC9A3R2 | Endothelial cells | 1.984 | 1.944 | 71.6 | 0.0E+00 |
| FLT1 | Endothelial cells | 1.835 | 1.801 | 70.5 | 0.0E+00 |
| PLVAP | Endothelial cells | 2.240 | 2.180 | 68.5 | 0.0E+00 |
| NOTCH4 | Endothelial cells | 1.469 | 1.439 | 66.9 | 0.0E+00 |
| HYAL2 | Endothelial cells | 1.535 | 1.484 | 65.9 | 0.0E+00 |
| EGFL7 | Endothelial cells | 1.384 | 1.348 | 65.5 | 0.0E+00 |
| HSPG2 | Endothelial cells | 1.616 | 1.565 | 65.3 | 0.0E+00 |
| ADGRL4 | Endothelial cells | 1.279 | 1.266 | 64.4 | 0.0E+00 |
| HBD | Erythrocytes | 1.337 | 1.335 | 48.1 | 0.0E+00 |
| SLC25A39 | Erythrocytes | 0.612 | 0.365 | 5.4 | 4.2E−18 |
| DCN | Fibroblasts | 4.164 | 4.151 | 97.5 | 0.0E+00 |
| FBLN1 | Fibroblasts | 2.614 | 2.590 | 96.1 | 0.0E+00 |
| CCDC80 | Fibroblasts | 2.769 | 2.738 | 93.8 | 0.0E+00 |
| PRELP | Fibroblasts | 2.012 | 2.000 | 88.6 | 0.0E+00 |
| ISLR | Fibroblasts | 1.633 | 1.625 | 87.4 | 0.0E+00 |
| LUM | Fibroblasts | 3.173 | 3.167 | 86.2 | 0.0E+00 |
| FBLN2 | Fibroblasts | 1.342 | 1.310 | 80.1 | 0.0E+00 |
| SFRP4 | Fibroblasts | 2.084 | 2.081 | 80.1 | 0.0E+00 |
| MFAP4 | Fibroblasts | 1.832 | 1.821 | 79.9 | 0.0E+00 |
| CTSK | Fibroblasts | 1.258 | 1.239 | 77.1 | 0.0E+00 |
| LYZ | Macrophages | 2.944 | 2.790 | 75.3 | 0.0E+00 |
| AIF1 | Macrophages | 2.203 | 2.114 | 74.0 | 0.0E+00 |
| LST1 | Macrophages | 1.842 | 1.769 | 70.3 | 0.0E+00 |
| MNDA | Macrophages | 1.226 | 1.205 | 60.6 | 0.0E+00 |
| MS4A7 | Macrophages | 1.265 | 1.240 | 60.4 | 0.0E+00 |
| MS4A6A | Macrophages | 1.409 | 1.358 | 58.0 | 0.0E+00 |
| SPI1 | Macrophages | 1.014 | 0.985 | 57.8 | 0.0E+00 |
| CSTA | Macrophages | 1.032 | 1.015 | 57.3 | 0.0E+00 |
| CLEC7A | Macrophages | 0.881 | 0.857 | 54.8 | 0.0E+00 |
| S100A9 | Macrophages | 1.888 | 1.757 | 54.2 | 0.0E+00 |
| TPSAB1 | Mast cells | 4.731 | 4.721 | 86.3 | 0.0E+00 |
| TPSB2 | Mast cells | 4.950 | 4.938 | 86.2 | 0.0E+00 |
| CPA3 | Mast cells | 2.597 | 2.594 | 77.2 | 0.0E+00 |
| MS4A2 | Mast cells | 1.827 | 1.825 | 65.9 | 0.0E+00 |
| HPGDS | Mast cells | 1.703 | 1.696 | 62.8 | 0.0E+00 |
| SLC18A2 | Mast cells | 1.424 | 1.423 | 57.3 | 0.0E+00 |
| VWA5A | Mast cells | 1.384 | 1.366 | 57.2 | 0.0E+00 |
| KIT | Mast cells | 1.337 | 1.328 | 57.1 | 0.0E+00 |
| GATA2 | Mast cells | 1.480 | 1.435 | 56.0 | 8.6E−214 |
| IL1RL1 | Mast cells | 1.336 | 1.294 | 55.6 | 3.1E−290 |
| KLRD1 | NK cells | 2.550 | 2.479 | 87.2 | 0.0E+00 |
| GNLY | NK cells | 3.413 | 3.254 | 77.2 | 0.0E+00 |
| GZMB | NK cells | 2.157 | 2.094 | 72.5 | 0.0E+00 |
| PRF1 | NK cells | 1.916 | 1.804 | 69.3 | 0.0E+00 |
| FGFBP2 | NK cells | 1.411 | 1.398 | 57.4 | 0.0E+00 |
| KLRF1 | NK cells | 1.163 | 1.153 | 52.7 | 0.0E+00 |
| GZMH | NK cells | 1.395 | 1.295 | 52.4 | 0.0E+00 |
| PLAC8 | NK cells | 1.243 | 1.150 | 50.1 | 0.0E+00 |
| CLIC3 | NK cells | 1.086 | 1.044 | 47.4 | 0.0E+00 |
| GZMM | NK cells | 0.928 | 0.858 | 45.7 | 0.0E+00 |
| RGS5 | Pericytes | 4.598 | 4.376 | 82.8 | 0.0E+00 |
| PLN | Pericytes | 1.948 | 1.917 | 75.9 | 0.0E+00 |
| TPPP3 | Pericytes | 1.638 | 1.587 | 69.8 | 0.0E+00 |
| GJA4 | Pericytes | 1.685 | 1.592 | 69.2 | 0.0E+00 |
| MYH11 | Pericytes | 1.426 | 1.394 | 62.1 | 0.0E+00 |
| NOTCH3 | Pericytes | 0.943 | 0.919 | 56.1 | 0.0E+00 |
| ISYNA1 | Pericytes | 0.897 | 0.851 | 50.5 | 0.0E+00 |
| COX4I2 | Pericytes | 0.866 | 0.844 | 48.2 | 0.0E+00 |
| C11orf96 | Pericytes | 0.940 | 0.878 | 48.1 | 0.0E+00 |
| CLMN | Pericytes | 0.802 | 0.750 | 47.1 | 0.0E+00 |
| IGHG1 | Plasma cells | 5.571 | 5.423 | 88.4 | 0.0E+00 |
| IGHGP | Plasma cells | 2.161 | 2.143 | 88.4 | 0.0E+00 |
| IGHG2 | Plasma cells | 1.291 | 1.280 | 80.5 | 0.0E+00 |
| JSRP1 | Plasma cells | 1.197 | 1.188 | 80.2 | 0.0E+00 |
| IGLV2-23 | Plasma cells | 1.272 | 1.253 | 80.0 | 0.0E+00 |
| ST6GAL1 | Plasma cells | 1.653 | 1.554 | 77.0 | 0.0E+00 |
| ITGB7 | Plasma cells | 1.726 | 1.645 | 76.9 | 0.0E+00 |
| MEI1 | Plasma cells | 0.833 | 0.791 | 71.1 | 0.0E+00 |
| VSIR | Plasma cells | 1.095 | 1.000 | 70.7 | 0.0E+00 |
| MAN1A1 | Plasma cells | 1.004 | 0.921 | 68.4 | 0.0E+00 |
| TRAC | T lymphocytes | 1.771 | 1.672 | 66.2 | 0.0E+00 |
| GZMK | T lymphocytes | 1.688 | 1.600 | 52.2 | 0.0E+00 |
| LTB | T lymphocytes | 1.110 | 1.044 | 46.1 | 0.0E+00 |
| DUSP4 | T lymphocytes | 0.888 | 0.816 | 34.2 | 0.0E+00 |
| KIAA1551 | T lymphocytes | 0.856 | 0.658 | 29.0 | 0.0E+00 |
| SPOCK2 | T lymphocytes | 0.691 | 0.558 | 28.5 | 0.0E+00 |
| CD8B | T lymphocytes | 0.635 | 0.599 | 28.0 | 0.0E+00 |
| LINC00152 | T lymphocytes | 0.668 | 0.548 | 27.8 | 0.0E+00 |
| GPR171 | T lymphocytes | 0.514 | 0.484 | 25.0 | 0.0E+00 |
| ALDOB | Tubular epithelial cells | 4.223 | 4.092 | 86.4 | 0.0E+00 |
| MIOX | Tubular epithelial cells | 3.262 | 3.150 | 77.7 | 0.0E+00 |
| HPD | Tubular epithelial cells | 2.237 | 2.192 | 65.7 | 0.0E+00 |
| PCK1 | Tubular epithelial cells | 2.075 | 2.033 | 61.7 | 0.0E+00 |
| ASS1 | Tubular epithelial cells | 2.087 | 1.995 | 60.5 | 0.0E+00 |
| FABP1 | Tubular epithelial cells | 1.960 | 1.928 | 59.7 | 0.0E+00 |
| MT1H | Tubular epithelial cells | 2.170 | 2.117 | 59.6 | 0.0E+00 |
| HRSP12 | Tubular epithelial cells | 1.767 | 1.715 | 58.6 | 0.0E+00 |
| FBP1 | Tubular epithelial cells | 1.835 | 1.732 | 53.5 | 0.0E+00 |
| ALDH6A1 | Tubular epithelial cells | 1.417 | 1.346 | 46.6 | 0.0E+00 |

Table 1 provides specific lists of marker genes representative of each cell cluster according to the present invention. The genes shown in Table 1 represent the markers having the most important impact for defining a cell cluster in the scRNA-seq data. The deconvolution tools use the expression value of genes over those pre-determined cell types (cell clusters) as a reference to estimate cell type proportions in bulk transcriptomics data (RNA-seq or BRB-seq data).

In the context of the present invention, the deconvolution step based on bulk expression data predicts the proportions of the above-mentioned cell clusters. The method according to the present invention thus comprises determining the proportion of each cell population corresponding to the 18 cell clusters described in a bulk tumor sample so as to evaluate which cell clusters are present in which proportion in the tumor sample.

The prognosis, i.e., in the context of the present invention, the expected development of ccRCC in the patient, is then determined based on said proportion.

Said proportion can be e.g. compared to reference values determined in a control population associated with a specific prognosis. Alternatively, the prognosis can be provided based on the proportion of cells belonging to one of the above specific cell clusters as compared to either the total amount of cells or to the total amount of cells in a specific cluster.

Typically, the proportion of cells belonging to each of the above-mentioned 18 cell clusters is provided by means of a percentage. Said percentage can be expressed with respect to the total amount of cells in the tumor/tumor sample or, alternatively with respect to the amount of cells belonging to another cluster or another group of cell clusters (e.g. the total amount of cells belonging to malignant cell clusters). The percentage can thus be provided as an absolute or as a relative value.

According to a specific embodiment, the proportion of cells belonging to each of the above-mentioned 18 cell clusters can also be expressed by means of a ratio of the total amount of cells belonging to one of the cell clusters to the total amount of cells belonging to another of the cell clusters. For instance, the proportion can be expressed as the ratio of the number of cells belonging to the Malignant cells_c8 cluster to the number of cells belonging to the Malignant cells_c9 cluster and vice versa.

In the context of the present invention, cells belonging to the cluster referred to as Malignant cells_c8 are correlated to a bad prognosis. A large quantity of cells belonging to this cluster is thus associated with a bad prognosis, i.e. with high-grade tumors. By “large quantities”, it is herein referred to more than 25%, preferably 50%, even more preferably 75% of cells belonging to the Malignant cells_c8 cluster among the total amount of Malignant cells of the tumor sample. Alternatively, “large quantities” can also refer to more than 10%, preferably 25%, even more preferably 35% of cells belonging to the Malignant cells_c8 cluster among the total amount of cells in the tumor. Patients presenting large quantities of cells of the Malignant cells_c8 cluster will therefore be more likely to have aggressive tumors with a significantly reduced overall survival. They will therefore necessitate extensive follow-up, possibly an adjuvant treatment or a more aggressive treatment to prevent the metastasis development.

In the context of the present invention, cells belonging to the cluster referred to as Malignant cells_c9 are correlated to a good prognosis. A large quantity of cells belonging to this cluster is thus associated with a good prognosis, i.e. with low-grade tumors. By “large-quantities”, it is herein referred to more than 25%, preferably 50%, even more preferably 75% of cells belonging to the Malignant cells_c9 cluster among the total amount of Malignant cells of the tumor sample. Alternatively, “large quantities” can also refer to more than 10%, preferably 25%, even more preferably 35% of cells belonging to the Malignant cells_c9 cluster among the total amount of cells in the tumor. Patients presenting large quantities of cells of the Malignant cells_c9 cluster will therefore be more likely to have good overall survival and will not necessitate extensive follow-up.

The “patient” according to the present invention is a human.

The invention will now be further illustrated by means of the following examples. These examples are provided for illustrative purposes only and cannot be interpreted as limiting the scope of the invention.

### EXAMPLES

### Patients and Methods

### Tumor Specimens

Fresh tumor samples, ranging from pT1a to pT3b and ISUP 2 to 4, were collected from a total of five untreated patients who underwent either radical or partial nephrectomy for ccRCC at the urology department of the Rennes university-hospital center. Patients met the requirements of the local institutional ethics committee.

### Single-Cell Preparation, Library Preparation, Sequencing and Analysis

For each tumor, ~2-9 punches were pooled and dissociated, and a partial depletion of ICs was performed to enrich the samples in MCs. Construction of single-cell libraries (Chromium™ Single Cell 3′ Library & Gel Bead Kit v2) was carried out according to the manufacturer's instructions (10× Genomics). Single-cell libraries were quality-controlled using the Agilent 2100 Bioanalyzer system and sequenced on an Illumina HiSeq 4000 instrument using a 2×100 paired-end sequencing protocol (Illumina, San Diego, CA). Data processing was performed in R as extensively detailed in Supplementary methods.

### Results

### Validation of the Experimental Procedure

We studied five samples (designated RCC1-5) each from separate untreated ccRCC patients with various clinical features, as well as one sample from a vena cava tumor thrombus from the RCC5 patient (RCC5_t) and a sample of RCC4 dissociated cells that were frozen (RCC4_f). As our primary objective was to build a single-cell atlas of predominantly MCs, we designed and evaluated, by flow cytometry, a dedicated protocol that showed good dissociation, viability and cell type. Importantly, it allowed us to enrich the MC content of the samples by dramatically reducing the proportion of immune cells, and proved to only slightly affect viability in frozen dissociated cells.

a Single-Cell Atlas of ccRCCs Composed of ~55,000 Cells

After confirming our ability to efficiently dissociate the tumor and enrich MCs, we sequenced the transcriptome of individual cells of seven samples (RCC1-5, RCC4_f and RCC5_t). Our dataset was complemented with three pairs of matched healthy renal and ccRCC tumor tissues (termed RCC [6-8] _n and RCC [6-8], respectively) [Young et al., Science 2018; 361:594-9]. Following data processing, a pseudobulk analysis revealed differences between both datasets, prompting us to perform a batch effect correction. The final scRNA-seq dataset was composed of 54,812 cells which were projected on 2D UMAP spaces.

Single-Cell RNA-Seq Uncovers the Cellular Composition in ccRCC

Cells were next partitioned into 35 cell clusters (termed c1-c35) which were associated with eight broad cell types based on specific marker genes such as CA9 and NNMT for MCs [Baniak et al., Histopathology 2020; 77:659-66; Tang. Carcinogenesis 2011; 32:138-45]. The balance between ICs and MCs across samples clearly distinguished both datasets, stressing the relevance of our approach to enrich MCs. Remaining ICs in RCC4 and RCC4_f were CD45-(PTPRC-) B/plasma cells, which explains their insensitivity to IC depletion. To further characterize MCs, we inferred their copy number variations (CNV) which confirmed the expected 3p loss in all samples and revealed sample-specific CNVs. Overall, these results confirm that ccRCC exhibits both intra- and inter-tumor heterogeneity.

### Malignant Cells Display Clonal Diversity at Chromosomal and Gene Expression Levels

To deepen the analysis of MC diversity, we next focused the analysis on the 15,471 MCs and the 3,737 tubular epithelial cells (or TECs; the normal cells from which MCs originate) which were subsequently partitioned into 24 clusters (termed c1-c24). With the exception of TECs (c1, c12 and c15), the vast majority of cell clusters did not mix across samples suggesting that each tumor had its own evolutionary history, as highlighted by the inter-tumor heterogeneity in chromosomal aberrations. Tumors also harbored various degrees of eITH. In particular in RCC2, MCs were distributed in five well delineated clusters; c3 (shared with RCC1, RCC3 and RCC4), c10, c23, c9 and c8, whereas other samples had a more homogeneous composition. Interestingly, the CNV analysis also revealed that the RCC2 sample carried higher intra-tumor CNV heterogeneity than the other tumors, in particular regarding 3q, 6p, 9q, 14q and 18p, that were lost in c8 but had contrasted status in other RCC2 malignant clusters. With the exception of c16 and c24, the cell cycle analysis revealed that all clusters were composed of a vast majority of cells in G1 and 9-36% in G2M, in accordance with typical observation in solid tumors (Terz et al., Cancer 1977; 40:1462-70 . . . . Along with CA9 and NNMT, we uncovered specific markers of MCs such as EGLN3, CP, MT3, NPTX2 and TRIB3. Expression of cell cycle markers (CKS2, CKAP2L and TOP2A) confirmed the cycling status of c16. As expected, we also found high expression of TEC markers such as ALDOB, MIOX, QDPR and GATM in c1, c12 and c15. We also noticed the expression of PTPRC and CD34 in two small cell clusters (c24 and c22, respectively) corresponding to residual contaminants by immune cells and endothelial cells, respectively.

### RCC2 Tumor Clonal Lineage Recapitulates Important Steps of Carcinogenesis

Given the variable degree of eITH observed in each tumor, we next tried to reconstruct tumor lineages within each sample. RCC2 displayed the longest cellular trajectory going from c3 through c10, c23 and c9, up to c8. The chromosomal imbalance analysis revealed that the CNV burden increased along the pseudotime, indicating progressive and cumulative chromosomal alterations, which provides additional elements in favor of a c3 to c8 lineage progression. Fifty-two regions were found to be significantly gained or lost along the RCC2 pseudotime, which was further supported by a phylogenetic analysis. We also found 2,229 differentially expressed genes (DEGs) across RCC2 MC clusters and tubular epithelial cells which were subsequently partitioned into nine expression patterns (termed P1-9). The functional analysis demonstrated the accumulation of aggressive features along the RCC2 lineage, with a notable peak in c8, related to epithelial-mesenchymal transition (EMT, a crucial initial stage of the metastatic process), angiogenesis, cell migration and cell adhesion. Overall these results suggest that MCs from RCC2 are linked together through a common tumor clonal lineage from indolent (c3) to aggressive (c8) MC clusters showing the sequential acquisition of typical molecular features corresponding to important steps of tumor progression.

Deconvolution of Specific Malignant Clusters is Associated with Tumor Stages and Survival in Low-Risk Patients

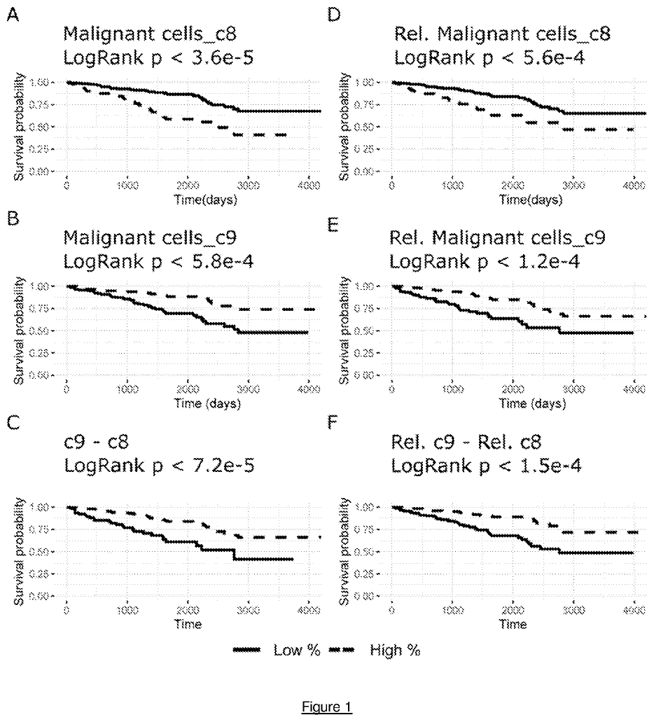

We next hypothesized that the RCC2 lineage could represent an in vivo model of ccRCC carcinogenesis which could be used as a reference. To verify this, we deconvolved the proportions of somatic cell types and RCC2 MC clusters on the TCGA-KIRC bulk RNA-seq dataset (525 samples) of ccRCCs. Substantial proportions of clusters c8 and c9 as well as non-immune microenvironment cells (endothelial cells, pericytes, myofibroblasts) were significantly detected, covering all four T stages. Despite their cellular proximity with MCs, TECs were not detected in ccRCC tumors, as expected. Concerning MCs, the main deconvolved populations were c9 and c8 with opposite trends, by respectively decreasing and increasing with tumor stages. We then evaluated the ability of deconvolved cell types to predict survival in either all or in T1-T2 low-risk ccRCC patients of the TCGA-KIRC cohort. When considering all patients, we found a significant association with poor prognosis of c8, and, to a lesser extent, of myofibroblasts and B cells. Conversely, c9, c10 and endothelial cells were three excellent predictors of good prognosis. When looking more specifically at low-risk patients, strikingly, only c8, c9 and c10, and, to a lesser extent, endothelial cells were still associated with survival. In addition, enrichment analysis of recurrent genomic features in TCGA-KIRC cohort (SR, section 8) revealed that the proportion of c8 was significantly associated with unfavorable tumors (CC-e.3 subtype and high genome instability) and the proportions of c9/c10 with favorable tumors (CC-e.2 and low genome instability), including in low-risk patients (FIG. 1, panels A-F). However, contrary to the proportions and relative proportions of c8, c9 and c10, previous RCC genomic prognosis subtypes and genome instability could not further stratify low-risk patients into two prognostic subgroups.

### Discussion

On the basis of conventional diagnosis, a significant proportion of patients considered to be at low-risk will eventually relapse (Makhov et al., Mol Cancer Ther 2018; 17:1355-64; Parasramka et al. Eur Urol Focus 2016; 2:608-15). A major issue in the management of ccRCCs hence consists in improving patient stratification through early detection of those who will eventually relapse and could have benefited from specific surveillance and potential adjuvant treatments. Biomarkers can be used to stratify these patients, but they remain insufficient. Moreover, the differences between gene expression signatures and corresponding protein detection constitute an important gap for clinical diagnosis (Buccitelli et al., Nat Rev Genet 2020; 21:630-44; Gygi et al., Molecular and Cellular Biology 1999; 19:1720-30) prompting the use of alternative and complementary approaches, such as transcriptomics in future diagnostic tools. In this context, it remains important to improve our understanding of tumor progression in ccRCCs in order to enhance patient care and consider personalized therapeutic strategies. To address these issues, we used scRNA-seq to decipher eITH in ccRCCs, with a specific focus on the malignant compartment, hypothesizing that this information could notably contribute to improve the detection of low-risk progressors. Since published scRNA-seq datasets in ccRCCs are often focused on the characterization of the immune compartment (Bi et al., Cancer Cell 2021; 39:649-61.e5; Borcherding et al., Commun Biol 2021; 4:122; Braun et al., Cancer Cell 2021; 39:632-48.e8; Krishna et al., Cancer Cell 2021; 39:662-77.e6) we established an experimental procedure to enrich the fraction of MCs by reducing the fraction of ICs. We also examined several punches per tumor to better capture eITH as suggested by Gerlinger and colleagues Gerlinger et al., N Engl J Med 2012; 366:883-92). Samples from five ccRCC patients ranging from pT1a to pT3b enabled us to assemble an atlas composed of ~25,826 cells (including ~48.3% of MCs) which we complemented with 28,986 cells (including ~10.3% of MCs) published by Young et al. Despite common molecular events such as the truncal 3p loss, the analysis of MCs revealed an important heterogeneity across samples. Various degrees of eITH in the distinct samples were observed, from low (RCC1, 6 and 7) to very high (RCC2 and 5 to a lower extent). We also noticed that both the inter- and intra-tumor heterogeneities in the samples of Young et al were lower than in our dataset, possibly due to the difference in sequencing depths, the number of punches per tumor and/or the higher proportion of MCs in our samples. Those differences appear as potentially critical features to properly capture eITH. While the overall CNV detection also revealed differences between datasets and turns out to be informative in our analysis, it should be considered with caution as CNVs were inferred from gene expression data and not from gold standard DNA analysis techniques (FISH, aCGH). Together these results suggest that ITH is not necessarily the rule, as pointed out in hepatocellular carcinoma (Xue et al., Gastroenterology 2016; 150:998-1008).

Among the different patients, we could identify a unique tumor sample (RCC2), displaying a yet never observed eITH consisting of five distinct MC subpopulations. Those were connected through a common lineage characterized by the progressive acquisition of highly aggressive features related to EMT, angiogenesis, cell migration and cell adhesion, as well as specific chromosomal abnormalities. RCC2 was a localized and apparently low-grade tumor (pT2b) without clinical/histological markers of aggressiveness. Yet, the RCC2 patient is currently under surveillance after having developed a suspicious pancreatic nodule, a preferred site for ccRCC metastasis. We believe that the RCC2 MC lineage can therefore represent a valuable resource for the community as an in vivo and representative model of tumor progression.

A landmark study used cell type deconvolution from TCGA data to successfully determine three groups of ccRCC patients according to lymphocyte T cell states and demonstrated their significant association with prognosis (Hu et al., Mol Ther 2020; 28:1658-72). Here we used a similar approach based on MC subpopulations composing the RCC2 tumor. Our results proved useful to predict survival and confirmed previous findings encouraging the consideration of eITH to identify predictive biomarkers (Gulati et al., Eur Urol 2014; 66:936-48). The ability of this method to stratify ccRCC patients from bulk RNA-seq is remarkable, especially the capacity of the predicted proportion of c8 and c9 MC clusters to serve as poor and good prognostic factors, respectively. In c9, despite the expression of aggressive features, the marked organization of adaptive immunity seems to confer an overall good clinical outcome. Importantly, some deconvolved cell types constituting the tumor-microenvironment are already known to be associated with survival (Edeline et al., Hum Pathol 2012; 43:1982-90; Li et al., Cell Death Dis 2022; 13:415). Here, not only do we confirm these results, but we go further by obtaining refined and resolutive malignant signatures with superior predictive power, especially in low-risk patients. Furthermore, although significantly associated with relevant RCC genomic subtypes defined by Chen et al. (Cell Rep 2016; 14:2476-89), we demonstrated that our MC-deconvolved approach better stratify low-risk patients. Interestingly, the proportion of T1-T2 patients (~10%) with a relatively high proportion of c8 is similar to the proportions of clinically low-risk progressors (10-25%), suggesting a potential overlap between the predicted and actual populations with poor outcomes. Our results suggest that low-grade tumors exceeding ~25% of c8 cells warrant reinforced surveillance. Hence, we present here the first proof of concept for using MC expression signatures to improve clinical management of ccRCC patients. Importantly a tumor that better recapitulates the stages of carcinogenesis could also complement and/or update our current deconvolution-based strategy. In the future it will be interesting to develop nomograms including relevant MC clusters and compare their accuracy with other conventional nomograms such as one recently developed in ccRCCs, based on scRNA-seq data (Xia et al., Sci Rep 2022; 12:10973). Therefore, we strongly encourage the use of transcriptomics in future clinical routine, especially since low-cost technologies such as BRB-seq (Alpern et al. Genome Biol 2019; 20:71) are now available and easily deployable, in order to enable the use of such predictive strategies and develop a personalized medicine. In this study, thanks to the single-cell profiling of a rare tumor showing a high eITH, we have established a reliable stratification approach for low-risk patients based on the deconvolution of specific MC subpopulations which could contribute to improving patient care.

### CONCLUSION

We described eITH in ccRCCs and used this information to establish significant cell population-based prognostic signatures and better discriminate ccRCC patients. This approach has the potential to improve the stratification of clinically low-risk patients and their therapeutic management.

1. A method for treating clear cell renal cell carcinoma (ccRCC) in a patient, said method comprising the following steps:
a) obtaining a bulk transcriptomic analysis of a tumor sample obtained from said patient;
b) submitting said bulk transcriptomic analysis to a deconvolution step so as to determine the proportion, in the tumor sample, of cells belonging to each of the 18 cells clusters consisting of Malignant cells_c3, Malignant cells_c10, Malignant cells_c23, Malignant cells_c9, Malignant cells_c8, Malignant cells_others, B lymphocytes, Collecting duct epithelial cells, Endothelial cells, Erythrocytes, Fibroblasts, Macrophages, Mast cells, NK cells, Pericytes, Plasma cells, T lymphocytes and Tubular epithelial cells;
c) providing a prognosis based on said cell proportion and treating the patient accordingly.

2. The method according to claim 1, wherein said transcriptomic analysis is obtained by submitting the tumor sample to RNA sequencing (RNA-seq).

3. The method according to claim 1, wherein said transcriptomic analysis is obtained by submitting the tumor sample to bulk RNA barcoding and sequencing (BRB-seq).

4. The method according to claim 1, wherein the Malignant cells_c8 cluster is correlated to a bad prognosis.

5. The method according to claim 1, wherein the Malignant cells_c9 cluster is correlated to a good prognosis.

6. The method according to claim 1, wherein step b. consists in determining the percentage of cells belonging to each cell cluster.

7. The method according to claim 1, wherein the tumor is classified as a stage I or II tumor according to the TNM staging system.

8. The method according to claim 1, wherein step b. is performed by using the data deposited as accession number GSE224630 in the National Center for Biotechnology Information Gene Expression Omnibus (NCBI/GEO) as a reference for deconvolution.
